(19) United States
(12) Plant Patent Application Publication
(10) Pub. No.: US 2003/0217401 P1
(43) Pub. Date: Nov. 20, 2003

(54) 'Fulmar' willow

(76) Inventors: William Ernest Fulcher, Madison, NC (US); Patrick David Fulcher, Madison, NC (US)

Correspondence Address: William E. Fulcher, P.O. Box 122, Madison, NC, 27025, US

(21) Appl. No.: 10/042,065

(22) Filed: May 17, 2002

(51) Int. Cl. A01H 5/00

(52) U.S. Cl. PLT/216

(57) ABSTRACT

A new variety of Salix matsudana named ‘Fulmar’, has contorted leaves and branchlets. It has a short trunk with a broad, open, irregular crown of drooping branches. The young branches are yellow, changing to brown as they mature. It is easily propagated from cuttings of young stems.

### DESCRIPTION

We found a large weeping willow tree approximately 11 m tall and 31 cm DBH, growing in a backyard in Greensboro (Guilford County), N.C.

This tree has the pendulous branches of a weeping willow, as well as the contorted branches of a corkscrew willow. It resembles Salix matsudana G. Koidz, ‘Pekin willow’. It has characteristics of both S.matsudana ‘Tortuosa’ and S. matsudana ‘Pendula’. The branches of ‘Tortuosa’ grow vertically, forming a conical tree. ‘Pendula’ has the pendulous branches of a typical weeping willow.

The mature tree has a short trunk with a broad, open, irregular crown of weeping branches. The bark is fissured and gray-brown (RHS N200A). The branchlets have smooth, yellow bark (RHS N144B). The older branches become brown (RHSN153D). The immature tree (1-2 m tall), has a twisted stem with gray-yellow bark (RHS 162B).

The contorted leaves are simple and alternate with pinnate venation. There are no stipules. The apex is acuminate, the base is cuneate, and the surfaces are glabrous, The abaxial surface us light green (RHS 139C). The adaxial surface is a darker green (RHS 143A). The mature leaves are 14-16 mm wide and 1.5-2 cm long with apetiole 2-3 mm long.

The mature tree has staminate catkins appearing with the leaves.

This fast growing tree is easily propagated from young stem cuttings 15-20 cm long. Initially, stem cuttings were taken in February, 2000. The rooted cuttings were moved up to 1 gallon pots in April, 2000. The plants were moved up to 3 gallon pots in August, 2000.

This tree has enough different characters from known willows that we propose this as a new variety of Salix matsudana which we will name ‘Fulmar’ willow.

This new variety has potential as a landscape feature when mature. It also has decorative characters when young. The contorted branches, when dried, can be used as features in flower arrangements.

### LIST OF FIGURES

FIG. 1 Mature tree, 11 m tall, 30 cm DBH.

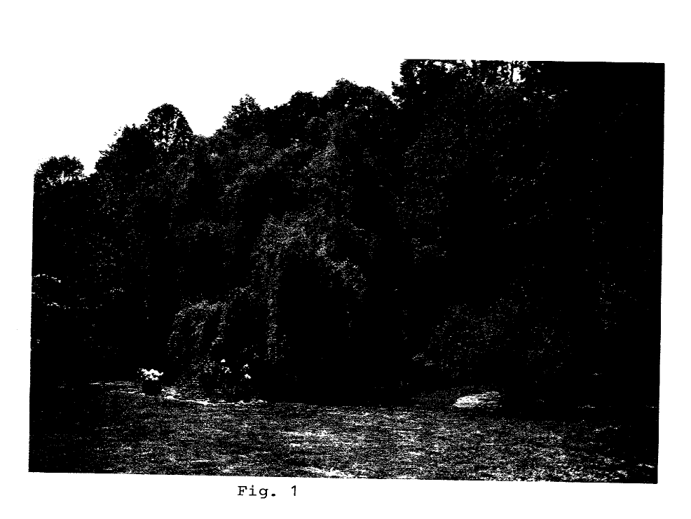

FIG. 2 Bark on mature tree.

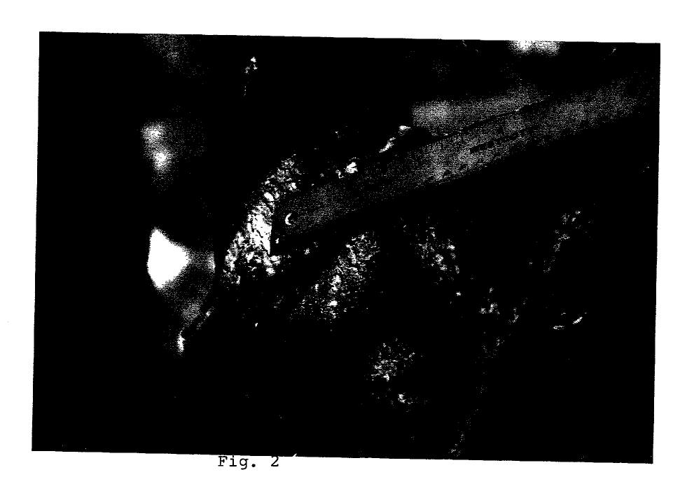

FIG. 3 Mature branches in winter.

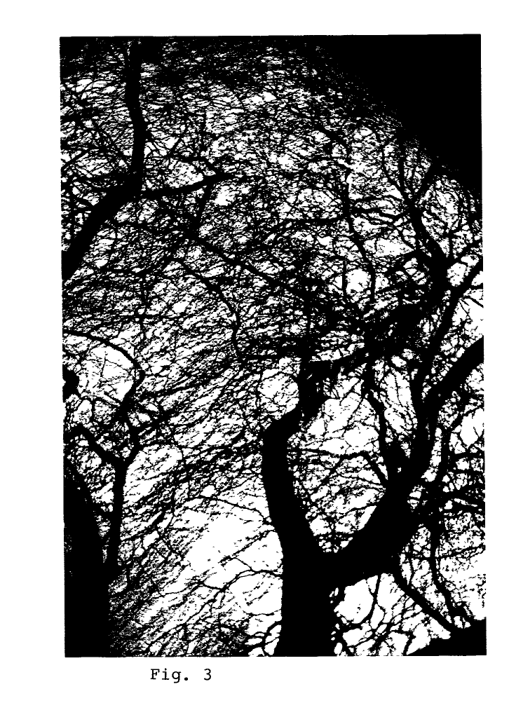

FIG. 4 Branchlets on mature tree.

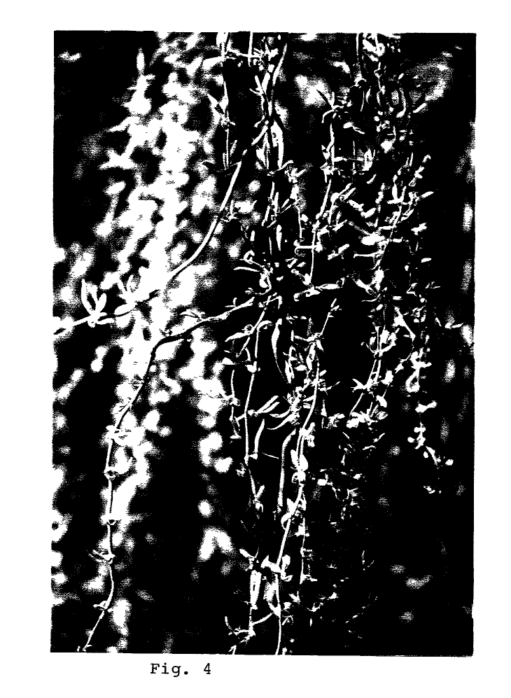

FIG. 5 Branchlets with staminate flowers.

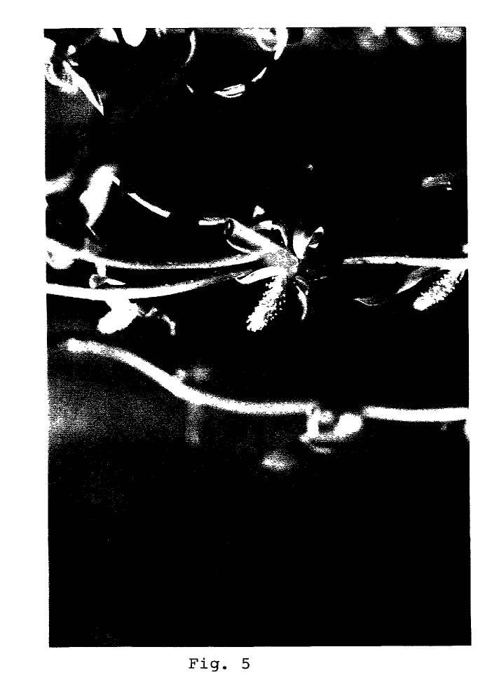

FIG. 6 Mature leaves.

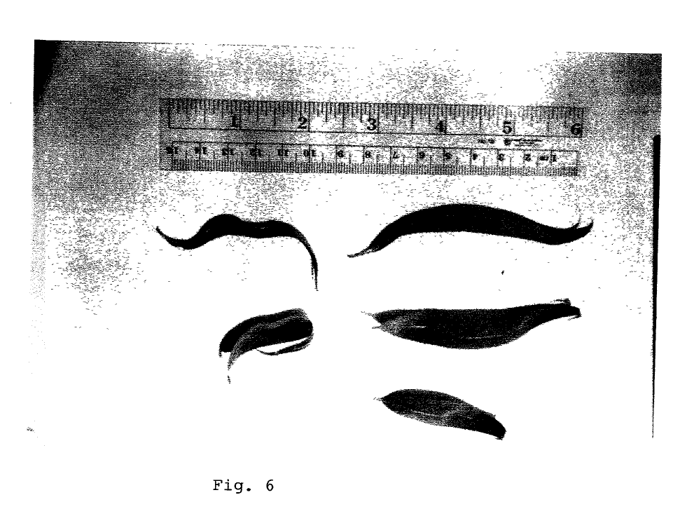

FIG. 7 Plant 18 months old in 3 gal. pot.

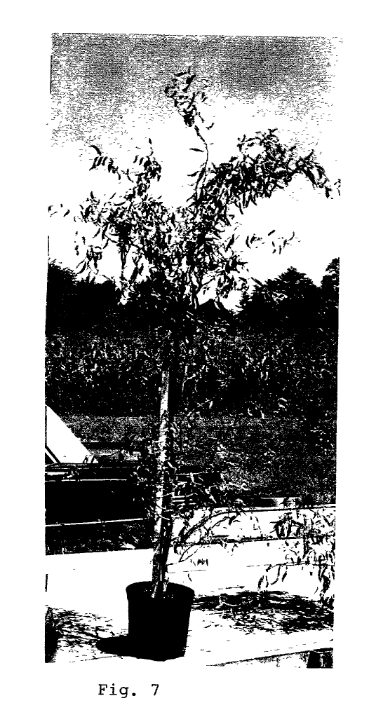

### REFERENCES

Brickell, Christopher Ed. 1989, Gardeners Encyclopedia of Plants and Flowers. Dorling Kindersley, London.

Hillier, Arthur 1974, Hilliers' Manual of Trees and Shrubs. Hillier and Sons, Winchester, England.

The Royal Horticultural Society 2001, RHS Colour Chart. RHS, London,England.

Staff of the L. H. Bailey Hortorium 1976, Hortus Third. Macmillan, New York.

I claim:

1. A new and distinct variety of Salix matsudana substantially as shown herein and described, characterized particularly by its unusual growth habit and pendulous, twisted (corkscrew) branches.
